(19) United States
(12) Patent Application Publication
(10) Pub. No.: US 2025/0122469 A1
(43) Pub. Date: Apr. 17, 2025

(54) SYSTEM AND METHODS FOR MANUFACTURING IN VITRO HIGH PERFORMANCE WOOD

(71) Applicant: Georgia Tech Research Corporation, Atlanta, GA (US)

(72) Inventors: Christos Athanasiou, Atlanta, GA (US); Cyrus K. Aidun, Atlanta, GA (US); Else-Marie Ulrika Egertsdotter, Atlanta, GA (US); Patritsia Stathatou, Atlanta, GA (US)

(21) Appl. No.: 18/917,678

(22) Filed: Oct. 16, 2024

Related U.S. Application Data
(60) Provisional application No. 63/544,270, filed on Oct. 16, 2023.

(51) Int. Cl. C12N 5/04; B29C 64/10; B33Y 10/00; B33Y 70/00; C12N 5/00

(52) U.S. Cl. CPC C12N 5/04; B29C 64/10; C12N 5/0025; B33Y 10/00; B33Y 70/00

(57) ABSTRACT

An exemplary embodiment of the present disclosure provides a method for generating in vitro wood. The method can comprise culturing a plurality of wood forming plant cells; encapsulating the plurality of wood forming plant cells in one or more hydrogels; developing one or more bioinks; and generating structures comprising the one or more bioinks. The one or more bioinks can comprise one or more plant cell culture medium components. In an exemplary embodiment, the plurality of wood forming plant cells are cambial meristematic cells, and can include genetically modified plant cells. The one or more bioinks can be configured to be loaded into a bioprinting system, which can generate the structures via an extrusion based method. The generated structures can be incubated to induce wood formation, monitored, and tested for superior material properties.

### CROSS-REFERENCE TO RELATED APPLICATIONS

This application claims priority and the benefit under 35 U.S.C. § 119 (e) of U.S. Provisional Patent Application No. 63/544,270, filed 16 Oct. 2023, of which the entire contents and substance are hereby incorporated by reference as if fully set forth below.

### FIELD OF THE DISCLOSURE

The various embodiments of the present disclosure relate generally to systems and methods for manufacturing in vitro wood, and more particularly to using plant cell culture technology with additive manufacturing to create in vitro near-net-shaped high performance wood.

### BACKGROUND

As natural resources dwindle and greenhouse gas emissions (GHG) increase, the use for renewable materials becomes urgent. Wood is an ideal candidate for sustainable societies and economies, as it is biobased, biodegradable, and absorbs carbon dioxide (CO₂) during its growth. Indeed, wood has a long history as a construction material and could potentially replace synthetic materials in increasingly ambitious modern structures, such as skyscrapers and wind turbines. However, the quality of natural wood varies greatly with seasonality, age, environmental conditions, and geographic location even among the same tree species. Low reliability of natural wood limits its use and could include additional processing and application of synthetic adhesives. Additionally, the current wood manufacturing process of reducing trees to lumber and then to laminates, is inefficient and wasteful. Demand for wood far exceeds supply, making current production and consumption rates unsustainable. Some embodiments of the present disclosure provide to address these challenges, by growing wood of consistent quality in desired forms.

During the last years there have been significant efforts to control and improve the mechanical performance of wood, through its delignification and densification. However, these methods involve chemical treatment and hot-pressing which do not allow for the creation of complex, monolithic 3D shapes, hence, constraining the scalability of the produced material. Recently, scientists managed to generate plant-based tissues in vitro in 3D bioprinted net-shape forms. Although 3D bioprinting of plant cells allows the development of complex plant tissue structures enabling scalability, efforts to-date use herbaceous plant cells which do not have the wood formation capabilities that trees have (e.g., Zinnia elegans cells), and develop plant-based end materials of poor mechanical properties.

Accordingly, there is a need for improved bioprinting methods for the production of wooden structures. As disclosed herein, various embodiments of the present disclosure provide such methods.

### BRIEF SUMMARY

An exemplary embodiment of the present disclosure provides a bioink for bioprinting including a plurality of wood forming plant cells; one or more hydrogels encapsulating the plurality of wood forming plant cells; and plant cell culture medium components.

In any of the embodiments disclosed herein, the plurality of wood forming plant cells can be cambial meristematic cells (CMCs).

In any of the embodiments disclosed herein, the CMCs can be derived from Populus sp. trees, Pinus taeda trees, Paulownia sp. trees, or any combination thereof.

In any of the embodiments disclosed herein, the plurality of wood forming plant cells can include genetically modified plant cells.

In any of the embodiments disclosed herein, the genetically modified plant cells can include one or more of lignin mutant plant cells, hemicellulose mutant plant cells, and cellulose mutant plant cells.

In any of the embodiments disclosed herein, the one or more hydrogels can include alginate, gelatin (GelMA), agarose, Gelzan, poly(ethylene glycol) (PEG), pluronic, Carboxymethyl Cellulose, or any combination thereof.

In any of the embodiments disclosed herein, the plant cell culture medium components can include nutrients, carbohydrates, growth regulators, or any combination thereof.

In any of the embodiments disclosed herein, the bioink can be configured to be loaded into a bioprinting system.

In any of the embodiments disclosed herein, the bioink can include a bioink formula. The bioink formula can be optimized using a surrogate machine learning model.

An exemplary embodiment of the present disclosure provides a method of generating wood in vitro including culturing a plurality of wood forming plant cells; encapsulating the plurality of wood forming plant cells in one or more hydrogels to form a plurality of encapsulated wood forming plant cells; developing one or more bioinks from the plurality of encapsulated wood forming plant cells; and generating structures including the one or more bioinks. The one or more bioinks can include one or more plant cell culture medium components.

In any of the embodiments disclosed herein, the method can further include incubating the structures to induce wood formation.

In any of the embodiments disclosed herein, the method can further include monitoring the incubated structures via imaging, confocal imaging, X-ray computed tomography, contrast-enhanced 3D micro-CT scanning, or any combination thereof.

In any of the embodiments disclosed herein, the method can further include determining one or more physical properties of the incubated structures. The one or more physical properties can be selected from the group consisting of tensile strength, flexural strength, yield strength, density, hardness, compressive strength, and impact strength.

In any of the embodiments disclosed herein, the method can further include determining, based at least in part on the one or more physical properties, a desired set of process parameters for generating wood.

In any of the embodiments disclosed herein, the desired set of process parameters can include one or more selected from the group consisting of wood forming plant cell type, hydrogel type, incubation time, bioprinting humidity, bioprinting pressure, bioprinting velocity, bioprinting temperature, infill density, and bioprinting needle tip diameter.

In any of the embodiments disclosed herein, the generated wood can have a tensile strength of between 80 and 300 MPa.

In any of the embodiments disclosed herein, generating the structures can include bioprinting the bioinks via an extrusion-based method.

In any of the embodiments disclosed herein, values of the desired set of process parameters can be determined via a surrogate machine learning model (MLM). The surrogate MLM can be trained based at least in part on images obtained during an incubation process and one or more physical properties of the generated wood.

In any of the embodiments disclosed herein, the structures can be generated with a predetermined shape based on an intended application of the generated wood.

In any of the embodiments disclosed herein, the one or more hydrogels can have a storage modulus of between 3 and 5 kPa.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides a block diagram of a method of generating wood in vitro, in accordance with some embodiments of the present disclosure.

FIG. 2 provides an image of an exemplary process for in vitro, local manufacturing of wood with improved mechanical properties, in accordance with some embodiments of the present disclosure.

FIG. 3 provides an image of an overview of experimental procedures for the development of effective CMC cultures, in accordance with some embodiments of the present disclosure.

FIG. 4 provides an image of an overview of experimental procedures for establishing genetically engineered Arabidopsis thaliana (ARA) cell cultures, in accordance with some embodiments of the present disclosure.

FIGS. 5A-C illustrate an exemplary 3D bioprinting approach, in accordance with some embodiments of the present disclosure. FIG. 5A illustrates a 3D bioprinter that can be used for 3D cultures. FIG. 5B details an exemplary syringe and exemplary sensors. FIG. 5C illustrates an enlarged optical image showing a cell's distribution in the bioprinted construct.

FIGS. 6A-C provide images of exemplary femtosecond laser-operated devices enabling high throughput mechanical testing of tenths of small-scale specimens in parallel, in accordance with some embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Various systems and methods are disclosed and will now be described.

The present disclosure creates near-net-shape, lab-grown, high-performance wood for advanced structural applications, by leveraging advancements in plant cell culture technology, synthetic biology, and additive manufacturing. In contrast to the traditional, wasteful, and polluting wood manufacturing processes embodiments of the present disclosure provides in vitro wood manufacturing techniques, that allow for the on-demand generation of wood structures, independent of climatic conditions and arable land availability, while avoiding waste generation and unburdening forests. The approaches disclosed herein can enable the production of wood with customized shape and functionality based on application and can provide a platform for discovery of new, previously unattainable applications for in vitro wood material.

To accomplish this, the present application discloses a novel in vitro wood formation process via two parallel processes: 1) Developing fast-growing cambial cell cultures induced to form wood cell structures within three-dimensional (3D) printed hydrogel scaffolds, wherein the hydrogel scaffolds can provide the desired final shapes, while enhancing cell growth, as they can be enriched with nutrients and growth regulators; and 2) Testing how alterations in lignin biosynthesis in genetically modified cells can affect the mechanical properties of the developed tissues. Alterations of lignin concentrations and compositions in model plant cells can be linked with impacts on the mechanical properties of the developed 3D bioprinted constructs. The findings from the two processes can be combined to use genetically engineered, fast-growing and stable cambial cell cultures induced to form wood of improved mechanical properties compared to its natural counterpart, within 3D bioprinted hydrogel scaffolds.

Recent breakthroughs in genetic engineering (e.g., CRISPR-Cas9), improved genome sequencing capabilities and cell culture techniques, together with advances in additive manufacturing have made the embodiments disclosed herein feasible. These advancements have enabled precise modifications of plant cell properties and the creation of customized 3D scaffolds that can provide a nutrient-rich environment for the growth of in vitro wood cells. This innovative approach offers significant potential for sustainable and controlled production of wood-based materials.

Embodiments of the present disclosure can leverage plant cell culture technology and genetic engineering to tailor lignin contents for 3D bioprinting structures. Additionally, high throughput mechanical characterization can be utilized for evaluating and optimizing structural performance, automation can be utilized for scaling up incubation and manufacturing, and data-driven modeling can be utilized for predicting material properties.

Here, the present application discloses a manufacturing approach that can yield high-performance wood in vitro, with the added advantage of scalability, providing the best of both worlds. Some embodiments of the present disclosure establish fast-growing cambial cell cultures, derived from the wood-forming tissues of trees, that can differentiate into the cellular structures of wood, after being encapsulated in hydrogels via 3D bioprinting. Therefore, complex, final shapes of in vitro wood can be developed. In parallel, the effect of lignin levels and composition of the plant cell wall on the mechanical properties of the 3D bioprinted constructs can be investigated, using existing mutations in model plant cell systems. The knowledge obtained from these two processes can be combined to alter lignin biosynthesis in wood-forming cambial cells that can be 3D bioprinted in complex shapes developing high performance in vitro wood.

The potential applications of the present disclosure can be multiple, from construction of wooden skyscrapers and wind turbines to microelectronics to in-space manufacturing. In some embodiments, wood can be generated in a predetermined shape selected based at least in part on the target application. For such applications to be successful it is desirable to develop wood that is reliable, high-performing, and accurately shaped. Some embodiments of the present disclosure allow for deployment in multiple scales, from mm to tens of meters. The design flexibility enabled by the in vitro approach allows for complicated 3D geometries of wooden building blocks that could be used to develop modular, large, complex lattice structures offering several advantages over conventional solid materials, including improved strength-to-weight ratios, increased energy absorption, and enhanced resistance to fatigue and impact. In addition, some embodiments of the present disclosure could be used to make 3D wooden printed circuit boards (PCBs) offering an eco-friendly and biodegradable alternative to traditional plastic PCBs, as well as for in-space manufacturing where lightweight, strong, and resistant to radiation materials are needed.

Also, in describing the disclosed technology, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, the disclosed technology can include from the one particular value and/or to the other particular value. Further, ranges described as being between a first value and a second value are inclusive of the first and second values. Likewise, ranges described as being from a first value and to a second value are inclusive of the first and second values.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

The components described hereinafter as making up various elements of the disclosed technology are intended to be illustrative and not restrictive. Many suitable components that would perform the same or similar functions as the components described herein are intended to be embraced within the scope of the disclosed technology. Such other components not described herein can include, but are not limited to, similar components that are developed after development of the presently disclosed subject matter.

As used herein, the term “structures” can be used to describe scaffolds, constructs, or other similar terms.

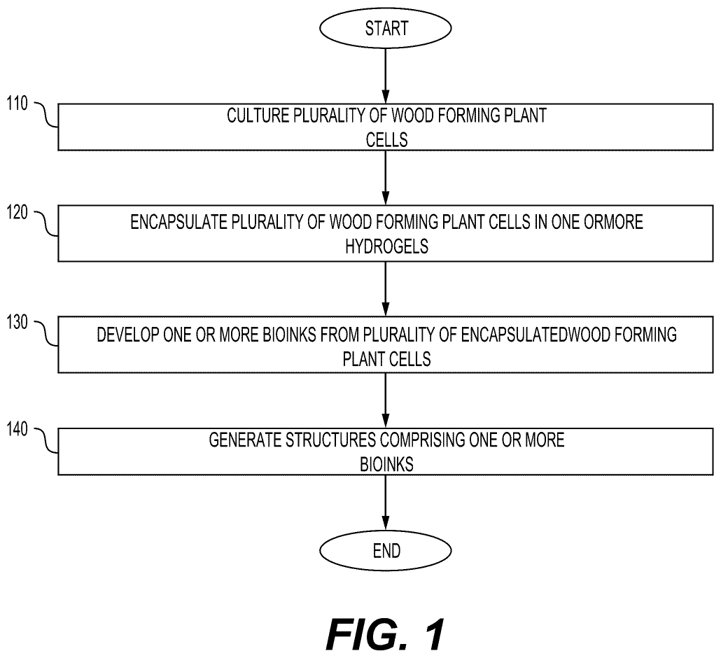

Referring now to the drawings, in which like numerals represent like elements, the present disclosure is herein described. FIG. 1 illustrates a method of generating wood in vitro. The method can include step 110: culturing a plurality of wood forming plant cells. As used herein, the term “wood forming plant cells” refers to cells derived from tissues of trees which have the inherent capability to differentiate into the cellular structures of wood through tracheary elements (TEs) formation. Culturing the plurality of wood forming plant cells, in some embodiments, can include harvesting the plurality of wood forming plant cells. As will be described in greater detail herein, harvesting the plurality of wood cells can include explanting tissue of a selected tree. For example, the plurality of wood forming plant cells can be cambial meristematic cells (CMCs). In some embodiments, the selected tree can be any of, but not limited to, Populus sp. trees, Pinus taeda trees, and Paulownia sp. trees. In some embodiments, culturing the plurality of wood forming plant cells can further include a surface sterilization. As will be appreciated, culturing the plurality of wood forming plant cells can be aimed at maximizing the formation of tracheary elements conducive to the generation of wood. The plurality of wood forming plant cells, in some embodiments, can include genetically modified plant cells. For example, genetically modified plant cells can include, but are not limited to, lignin mutant plant cells, hemicellulose mutant plant cells, cellulose mutant plant cells, or any combination thereof. Genetically modified cells can be characterized as having an altered cell wall composition. The cell wall composition, in some embodiments, can be altered based at least in part on desired physical properties of the generated wood.

The method can further include step 120: encapsulating the plurality of wood forming plant cells in one or more hydrogels. Encapsulating the plurality of wood forming plant cells can form a plurality of encapsulated wood forming plant cells, as will be appreciated. The one or more hydrogels can be selected from a group including, but not limited to, alginate, gelatin (GelMA), agarose, Gelzan, poly(ethylene glycol) (PEG), pluronic, and Carboxymethyl Cellulose. In some embodiments, the one or more hydrogels can include any combination of the group including alginate, gelatin (GelMA), agarose, Gelzan, poly(ethylene glycol) (PEG), pluronic, and Carboxymethyl Cellulose. The one or more hydrogels, in some embodiments, can be selected based at least in part on a cell type of the plurality of wood forming plant cells. The one or more hydrogels can be configured to promote wood formation in the plurality of wood forming plant cells. As will be described in greater detail herein, the one or more hydrogels can be enriched with nutrients, carbohydrates, growth regulators, or any combination thereof. In some embodiments, the one or more hydrogels can be enriched with plant cell culture medium components. The plant cell culture medium components can include nutrients, carbohydrates, growth regulators, or any combination thereof. As will be appreciated, the plant cell culture medium components can be combined with the plurality of encapsulated wood forming plant cells.

The method can further include step 130: developing one or more bioinks from the plurality of encapsulated wood forming plant cells. In some embodiments, the one or more bioinks can include the plurality of wood forming plant cells, the one or more hydrogels, and the plant cell culture medium components. In some embodiments, the one or more bioinks can include the plurality of encapsulated wood forming plant cells, the one or more hydrogels, and the plant cell culture medium components. The one or more bioinks, as will be appreciated, can be a medium which may be printed via an additive manufacturing process. That is, the one or more bioinks can be configured to be bioprinted. Thus, the one or more bioinks can be configured to be loaded into a bioprinting system. The bioprinting system can employ an extrusion-based printing method, as will be described in greater detail herein. The one or more bioinks can further include a bioink formula. The bioink formula, in some embodiments, can be optimized using a surrogate machine learning model (MLM). In some embodiments, optimization of the bioink formula can be based at least in part on the cell type, the desired physical properties, an application of the generated wood, or any combination thereof.

The method can further include step 140: generating structures comprising the one or more bioinks. As will be appreciated, generating the structures can include bioprinting the one or more bioinks. In some embodiments, the one or more bioinks can be bioprinted via an extrusion-based method. That is, in some embodiments, the structures can be generated using a computer-aided layer by layer deposition technique. The structures can be generated with a predetermined shape. In some embodiments, the predetermined shape can be based on an intended application of the generated wood. Intended applications are described in greater detail herein.

The method can further include incubating the structures to induce wood formation. Incubations conditions, that is, can be selected based at least in part on an ability to induce the formation of wood. Incubated structures, in some embodiments, can be monitored via imaging, confocal imaging, X-ray computed tomography, contrast-enhanced 3D micro-CT scanning, or any combination thereof. Monitoring of the incubated structures, as will be appreciated, can be used to determine one or more physical properties of the incubated structures. The one or more physical properties can be selected from a group consisting of tensile strength, flexural strength, yield strength, density, hardness, compressive strength, and impact strength. The one or more physical properties, as will be appreciated, can be selected based on the desired physical properties of the intended application. Values of the one or more physical properties, in some embodiments, can be used to determine the intended application. As will be described in greater detail herein, the values of the one or more physical properties can be determined via a high throughput mechanical testing process. In some embodiments, a desired set of process parameters can be determined in order to better perform the method in future iterations. For example, the desired set of process parameters can include one or more selected from the group consisting of wood forming plant cell type, hydrogel type, incubation time, bioprinting humidity, bioprinting pressure, bioprinting velocity, bioprinting temperature, infill density, and bioprinting needle tip diameter. The desired set of process parameters can be determined based at least in part on the one or more physical properties. Values of the desired set of process parameters can be determined via a surrogate MLM. In an example embodiment, the surrogate MLM can be trained based at least in part on one or more images obtained during an incubation process and one or more physical properties of the generated wood.

Details that enable the methods disclosed herein will now be described. As discussed below, one or more of the techniques disclosed herein can be combined to create techniques for the manufacture of wood structures tailored to a desired application.

Development of Wood Forming Cambial Meristematic Cell (CMC) Cultures. Wood Formation is a Complex Process Occurring Through a Series of Coordinated Processes.

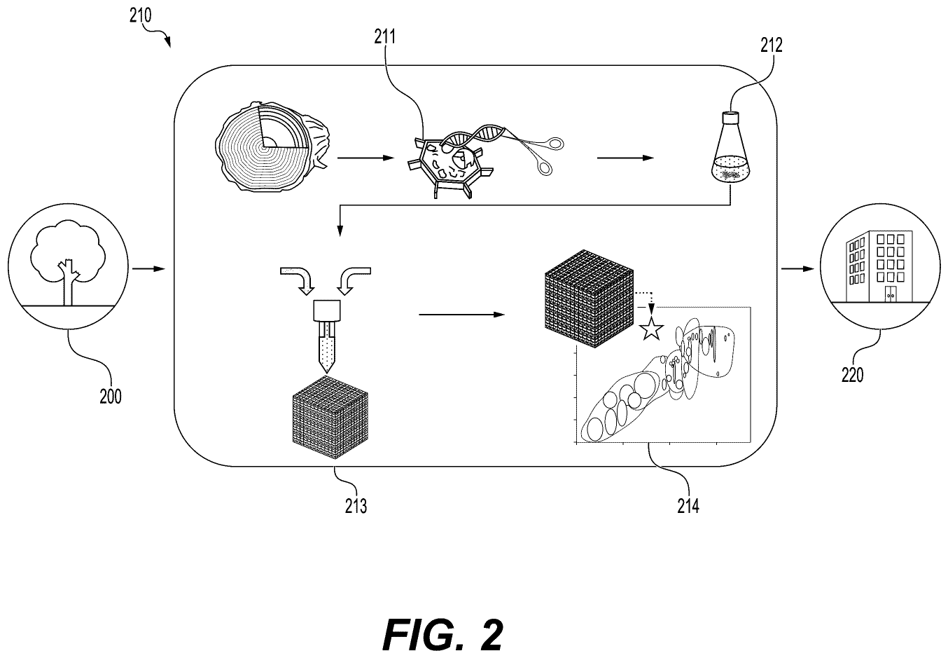

An example embodiment of a method of making net-shaped, in vitro, high performance wood is illustrated in FIG. 2. The method, at a high level, can include identifying trees 200 from where wood forming cells are to be harvested or selected; generating wood in vitro 210; and utilizing the generated wood in applications 220. The present disclosure focuses on methods for generating wood in vitro 210. Generating wood in vitro 210 can include harvesting a plurality of wood forming cells 211; culturing the plurality of wood cells 212; combining the plurality of wood cells with hydrogels to create a bioink and bioprinting the bioink into a structure 213; and generating wood within the bioprinted structure 214.

An example embodiment of the method of making net-shaped, in vitro, high performance wood can further include cultivating wood forming plant cells, developing bioinks by encapsulating the wood forming plant cells in one or more hydrogels, printing the bioinks into desired shapes that serve as three-dimensional (3D) scaffolds for cells to grow and differentiate; incubating the 3D bioprinted constructs in automated specialized control chambers; and evaluating the structural performance of the 3D bioprinted constructs during and after incubation using high-throughput mechanical characterization and data-driven modeling. Cultivating the wood forming plant cells, in a continued example embodiment, can include initiating in vitro cultures from wood forming tissue of a selected tree species; establishing optimal culture conditions for sustainable cell multiplication and wood formation; establishing methods for induction of wood formation in the cell cultures; and establishing methods for maintenance of wood formation in the cell cultures. In some embodiments, the one or more hydrogels can be enriched with nutrients, carbohydrates, and growth regulators to enhance cell growth. The plurality of wood forming plant cells can be defined as cells derived from tissues of trees which have the inherent capability to differentiate into the cellular structures of wood through tracheary elements (TEs) formation. Examples include, but are not limited to, cambial meristematic cells (CMCs), genetically modified cells (including genetically modified CMCs), and the like. For example, cells can be genetically modified to alter their cell wall compositions, which can include but are not limited to percentage makeup of lignin, hemicellulose, cellulose, etc. to achieve desired physical properties in the resulting wood structures.

In a continued example embodiment, the plurality of wood forming plant cells can be genetically modified cells with altered amounts and compositions of their cell wall components via regulation of their biosynthetic pathways. The tissue samples derived from trees, or, for example, the cambial tissue samples, can be surface sterilized before processing for starting the culture, and slices of the cambium can be laid on top of semi-solid induction media. Developing undifferentiated cells, or callus, can be transferred to a container with fresh medium once mm-scale diameter clumps of growing tissue are formed. Small pieces (100-500 mg) of the callus tissue can then be transferred to baffled flasks filled with liquid medium of the same composition. Suspension flasks can be kept in the dark at 22° C. on a rotary shaker table and subcultured weekly by transferring 10 mL of sedimented cells to 50 mL of fresh medium of the same composition. Further, the composition, structure, and relative abundance of TEs in the developed wood forming cell cultures can be observed using bright field and polarized microscopy, counting different cell types in a hemocytometer, or by applying different stains to visualize cell wall components via fluorescence microscopy. The genetically modified wood forming cells, in a continued example embodiment, can be harvested for analyzing cell wall components, contents, and compositions to confirm that the phenotypes associated with the mutant starting cells have carried over to the cell cultures using quantitative and qualitative gravimetric, spectrophotometric, and imaging techniques, which may include, but are not limited to, Klason gravimetric method, nitrobenzene oxidation, gas chromatography, and mass spectrometry.

As will be appreciated, the one or more hydrogels can be prepared at specific concentrations to match the properties of agar, or more specifically, with a storage modulus of 3-5 kPa. The one or more hydrogels, in some embodiments, can then be autoclaved and stored at suitable temperatures to prevent solidification before use. Concentrated liquid wood forming cell cultures, culture media, and growth regulators can be gently mixed with the one or more hydrogels, after preparation, to prepare the bioinks. In some embodiments, the one or more hydrogels can be in liquid form. Growth regulators can include but are not limited to auxin and cytokinin.

In a continued example embodiment, the bioinks, once developed, can be placed in 10 mL sterile luer-lock syringes and printed in 3D scaffolds via an extrusion-based bioprinting method. The bioprinting method, in some embodiments, can allow for the spatial patterning of cell-laden hydrogels using a computer-aided layer by layer deposition technique. In some embodiments, the bioinks can be formulated and printed inside a laminar flow hood under sterile conditions.

The 3D bioprinted constructs, or scaffolds, can be enclosed in a sterile container sealed with parafilm, and covered with the same culture medium solution, or plant cell culture medium components, used for the bioink preparation. The plant cell culture medium components can be configured to induce wood formation in the wood forming plant cells. In some embodiments, the plant cell culture medium components can induce differentiation of TEs within generated structures post-bioprinting. The plant cell culture medium components can comprise elements that are configured to enrich the hydrogels to be more conducive to wood formation. For example, the plant cell culture medium components can include, but are not limited to, nutrients, carbohydrates, growth regulators, or any combination thereof. The solution can be replaced together with the parafilm coverage at intervals allowing for optimal multiplication and for inducing wood formation processes. The 3D bioprinted constructs, in a continued example embodiment, can be incubated in the dark inside a specialized chamber for 1-3 months. The incubation chamber can be equipped with an automated climate control system for continuously monitoring and adjusting key environment factors, which include, but are not limited to, temperature, humidity, CO₂ levels, and air pressure. The temperature inside the chamber can be maintained within 18° C.-37° C., depending on the specific requirements of the constructs, with a relative humidity of 100% to prevent desiccation and promote cellular activity. The incubation chamber can be made from materials that ensure sterile conditions, which include but are not limited to stainless steel and coated aluminum, and can include an integrated HEPA filtration system to minimize contamination. The chamber may feature a digital control panel for easy monitoring and adjustment of environmental parameters, with data logging capabilities for tracking incubation conditions over time. In some embodiments, a fan-assisted airflow system can be employed to ensure uniform conditions throughout the chamber.

During the incubation period, as will be appreciated, cell growth and differentiation inside the bioprinted structures can be examined, or monitored, via imaging and X-ray computed tomography. Imaging can include, but is not limited to, confocal and TEM. X-ray computed tomography can include, but is not limited to, contrast-enhanced 3D micro-CT scanning.

In a continued example embodiment, a high throughput approach can be adopted to assess cell viability inside the bioprinted constructs at specific time intervals during incubation. The period of incubation can be 2 weeks. The high throughput approach can be adopted via bright field microscopy and confocal imaging. Bioink droplets ca be printed inside eight-well-chambered coverslips, which can facilitate cell imaging at multiple time points. The bioink, in some embodiments, can be printed inside petri dishes in mm scale beams of rectangular cross-sections to allow for mechanical testing. In a continued example embodiment, mechanical testing can include a surrogate machine learning model (MLM), which can be trained at least in part on images from confocal microscopy obtained during the incubation process.

The developed 3D bioprinted and incubated constructs can have superior mechanical properties compared to natural wood. In some embodiments, superior mechanical properties can include, but are not limited to, more than two times higher density, tensile strength, hardness, and toughness. In an exemplary embodiment, the generated wood can have a density of between 900 and 1,300 kg/m³. In another exemplary embodiment, the generated wood can have a tensile strength of between 80 and 300 MPa. Applications of the developed 3D bioprinted constructs can include, but are not limited to, wooden buildings, lattice structures and furniture, blades of wind turbines, microelectronics components, 3D wooden PCBs, packaging, and in-space manufacturing applications.

Justification, specific implementations, and alternative embodiments of the continued example embodiment are described in greater detail herein.

Tracheary elements, which are xylem cells specialized for facilitating water and solutes transportation throughout plants and trees, undergo a differentiation process which is crucial for enabling wood formation. TEs differentiation has been studied in vitro. Effective model plant cell systems, able to achieve up to 90% TEs formation in the lab, only exist for herbaceous plants, like Zinnia elegans, which cannot lead to wood formation. In vitro TEs formation from woody plants and trees is limited and less effective, typically ranging between 16-65%. In parallel, recent studies have demonstrated the ability to culture and maintain in the lab a particular type of cells from trees, which have the inherent capability of forming wood, i.e., the meristematic cells from cambial tissues. Such cells can develop the cellular structures of natural wood and can be maintained over prolonged times. However, cambial meristematic cell (CMC) cultures have not been explored as trans-differentiable cultures for TEs formation.

The present disclosure induces TEs formation in CMC cultures, combining the above approaches, for developing in vitro wood cellular structures. CMCs from different tree species can be used to cover a broad range of wood cellular structures, and protocols for controlling their in vitro proliferation and TEs differentiation processes can be developed by applying and testing different treatments and culture conditions. A first step includes developing standard culture conditions supported by solidified and subsequently liquid culture medium, followed by advanced culture techniques for cellular growth and development of cellular wood structures within 3D bioprinted constructs. The following tree species with different characteristics and potential can be considered: Poplar, a hardwood species for which the most advanced cell culture system and molecular tools exist, and relevant studies are available (small and fully sequenced genome, plethora of gene characterization studies); Loblolly pine, a softwood species which holds the greatest commercial significance among conifer species in the Southeast USA; Paulownia, which is an emerging hardwood species with high potential for carbon capture and agroforestry applications.

Several factors affecting CMC cultures, such as nutrient composition, plant growth regulators, pH, light, humidity and temperature, subculture intervals and incubation duration, can be explored and optimized for sustained and optimized cell culture proliferation and TEs formation. The culture process can comprise two phases, i.e., earlier cell growth in shaken liquid cultures to enable faster and uniform growth, followed by cell growth and differentiation for wood biosynthesis in 3D bioprinted scaffolds. Initiation and growth of CMC cultures can follow the methods described in Partap et al., 2022. The following actions can be deployed.

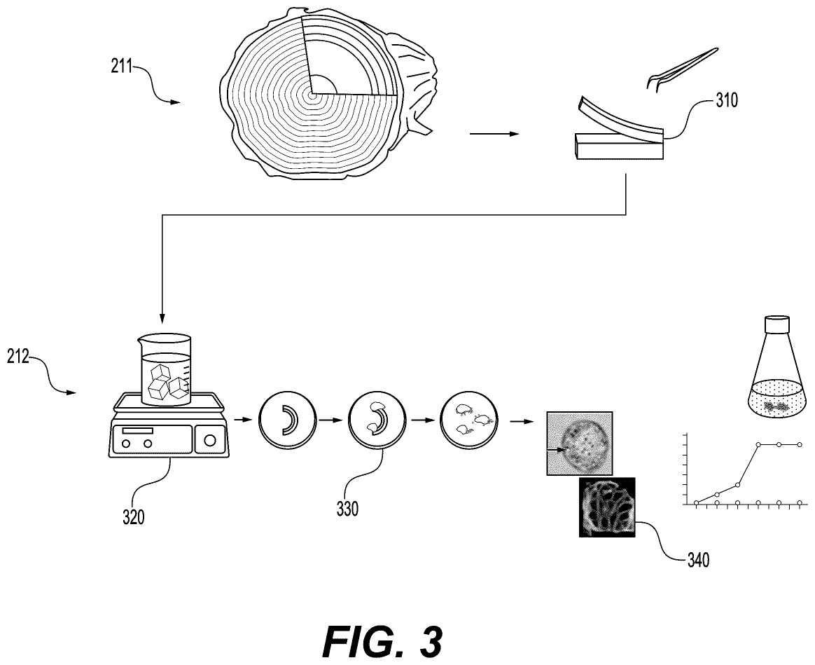

FIG. 3 illustrates an exemplary embodiment of harvesting the plurality of wood forming cells 211 and culturing the plurality of wood forming cells 212. Harvesting the plurality of wood forming cells can include identifying a cell type to be harvested. As will be appreciated, the cell type can be a CMC, which may be harvested from the cambium of a tree. Harvesting the plurality of wood forming cells 211 can include harvesting a cambium explant 310. Culturing the plurality of wood forming plant cells 212 can include surface sterilization 320; development of callus culture and optimization 330; and suspension of CMC culture for fast optimization and maximum TEs formation 340.

Initiation of cambial cell cultures from the selected tree species: Explants used to start the in vitro cambial cell cultures can be extracted from the live cambium of Poplar, Loblolly Pine, and Paulownia trees. The cambial tissue samples can be surface sterilized before processed for starting the culture, and slices of the cambium can be laid on top of semi-solid induction media. Media previously shown to be successful for similar species can be selected as the starting point and can be then optimized for the specific genotypes under consideration.

Establishment of culture conditions for CMC multiplication: Developing callus (undifferentiated cells) can be transferred to a container with fresh medium of the same composition with step 1, once 3-5 mm diameter clumps of growing tissue have been formed. Different compositions of culture media can be tested to optimize multiplication rates. Several subcultures can be developed to obtain 1-3 grams of callus. Small pieces (100-300 mg) of the callus tissue can be then transferred to 250 mL baffled Erlenmeyer flasks filled with 50 mL liquid medium of the same composition. Suspension flasks can be kept in the dark, at 22° C. on a rotary shaker table, and subcultured weekly by transferring 10 mL of sedimented cells to 50 mL of fresh medium of the same composition. The goal is to achieve a cellular division rate of approximately 150 times the increase in cell counts per week, as reported elsewhere.

Identification of suitable conditions for TEs formation in the cambial cell cultures: TEs differentiation and secondary cell-wall formation in cell cultures of coniferous gymnosperms or of hardwood trees, like hybrid poplars, can be tested on CMC cultures grown on semi-solid medium. Recently, CO₂ levels were shown to affect lignin biosynthesis in wood from Eucalyptus. The present disclosure can test if CO₂ levels affect TEs formation in the developed CMC cultures as well.

Assessment of the developed CMC cultures: The composition, structure, and relative abundance of TEs can be observed using bright field and polarized microscopy, counting different cell types in a hemocytometer, or by applying different stains to visualize cell wall components via fluorescence microscopy.

Once effective CMC cultures with high TEs formation levels are developed, the suspension cultures in flasks can be transferred to a bioreactor with controlled conditions to scale-up the CMC production process. The developed wood-forming CMC cultures can be then blended with nutrient rich hydrogels for 3D bioprinting.

Developing effective CMC culture systems that can be maintained over prolonged times, at a high rate of multiplication and with cells in a physiological state that can be induced to form TEs, is a challenging task. Therefore, the present disclosure includes working in parallel with the model plant cell culture system from Arabidopsis thaliana (ARA). ARA has the benefit of being the best studied model plant in terms of gene regulatory pathways including TEs formation. In addition, the ARA system has a small genome of only 132 megabase pairs (Mbp), for which an abundance of relevant molecular tools exists.

Risks and mitigation actions: Medium risk that CMC cultures are not fast multiplying and the TEs induction rate is low. To mitigate risks, developing and testing CMC cultures from different tree species can be used, while working with the ARA model system to be able to demonstrate all the disclosed tasks.

Establishing of Genetically Engineered ARA Cell Cultures with Altered Lignin Amounts and Compositions.

Lignin is a 3D polymer with a high molecular weight that accounts for 30% of the dry weight of plant cell walls and is available for their structural integrity. There have been many efforts to alter the lignin composition of plant cell walls to tune their mechanical properties. However, regulation of lignin biosynthetic pathways in plants is complex and occurs at many different levels, rendering it difficult to only target lignin without affecting other available cell wall compounds, such as cellulose. Consequently, there is contradicting evidence in the literature on whether altered lignin levels and compositions improve or deteriorate the mechanical properties of genetically engineered plant cells.

Establishing fast-growing wood-forming CMC cultures is a challenging and demanding task which could include at least 1 year of research and development. During this present disclosure, the present disclosure works with genetically engineered non-wood forming cells of the model plant Arabidopsis thaliana (ARA) and demonstrate that cell cultures with altered lignin biosynthesis can be utilized for making plant-based materials of improved mechanical properties. This can showcase the potential for future in vitro wood formation with tailored mechanical properties. The obtained insights can establish a relationship between altered lignin cell contents and mechanical properties of 3D bioprinted constructs made with the genetically engineered cells, and can guide future, similar genetic modifications of lignin in wood-forming cells.

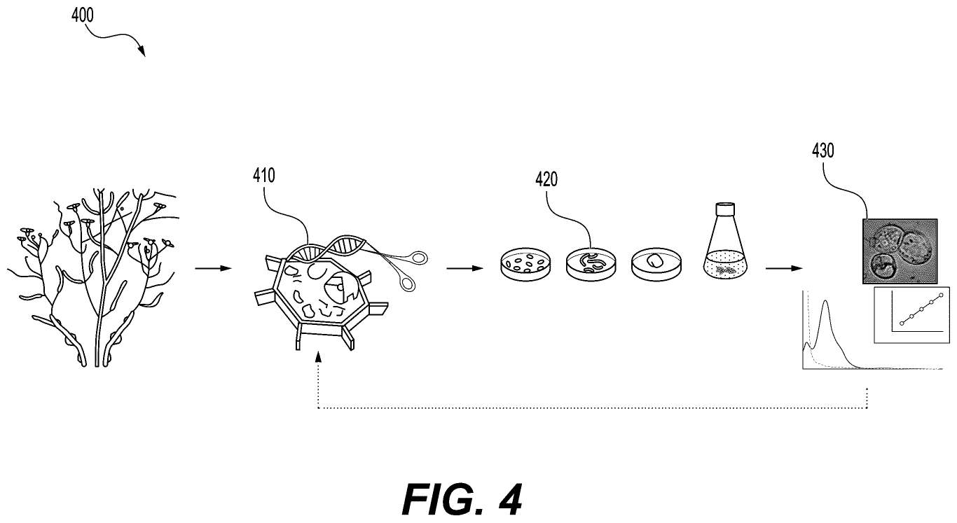

Several ARA mutants with altered lignin biosynthesis exist. This prompted the use of ARA as a model cell culture system for the generation of wood in vitro. An exemplary method for a model ARA cell culture system 400 is shown in FIG. 4. In some embodiments, the method for establishment of the model ARA cell culture system 400 can include harvesting of a plurality of ARA cells 410; development of ARA callus cultures 420; and analysis of lignin amount and composition 430. Harvesting of the plurality of ARA cells 410, as will be appreciated, can include identifying ARA cells with altered lignin amounts and compositions, such that the plurality of ARA cells may be genetically engineered.

The following methods, as illustrated in FIG. 4, can be further applied to establish the model ARA cell culture systems: Selection of mutant ARA cells: Seeds for control ARA cell cultures and ARA mutants with verified alterations in lignin content and composition can be obtained from the Arabidopsis Biological Resource Center. The present disclosure can select mutations with both lower and higher contents compared to wild types, as well as with different lignin compositions.

Development of callus and suspension cultures: Callus cultures can be obtained by the 90% effective induction of callus cultures from initial explants taken from plants grown from the control and mutant seeds. Fast-dividing suspensions cultures can be started from the callus cultures and maintained by subculture substantially every seven days in modified Murashige and Skoog (MS) medium supplemented with 1 μg/mL 2,4-Dichlorophenoxyacetic media and 3% sucrose, and subcultured on a rotary shaker at 120 rpm, in the dark at 22° C.

Induction of TEs formation: The induction of TEs in ARA suspension cultures has been described previously. Briefly, a 7.5-mL aliquot of 7-day-old subcultured cell suspension can be transferred into 42.5 mL of fresh medium including 1 μM brassinolide and 10 mM boric acid, and it can be then maintained on a rotary shaker at 120 rpm in the dark at 22° C.

Analysis of lignin levels & composition: The developed ARA cells can be harvested for analyzing lignin contents and compositions to confirm that the phenotypes associated with the mutant starting plants have carried over to the cell cultures. Various quantitative and qualitative gravimetric, spectrophotometric, and imaging techniques can be tested for this, including the Klason gravimetric method, nitrobenzene oxidation, gas chromatography and mass spectrometry (Py-GC/MS).

In some embodiments, immersion bioreactors can be used as part of the culturing process. The immersion bioreactors can make automated adjustments to cell cultures to ensure cell culture growth. An automated platform for cell culture growth may include a laminar flow hood, an imaging system, a deposition tray, and one or more deposition glass tubes. In some embodiments, the bioinks can be stored in a laminar flow hood. An imaging system can be employed to execute the imaging methods as discussed in greater detail herein.

The model cell culture system Arabidopsis Thaliana (ARA) can be used as a proxy for setting up the experimental pipeline. Although ARA is not a wood forming species, it is the best studied model plant in gene regulatory pathways and has a small genome of only 132 megabase pairs (Mbp), for which an abundance of relevant molecular tools exist. There are also many ARA mutant lines available with demonstrated altered lignin contents. Suspension cultures and callus cultures of light grown and dark grown ARA cells can be rapidly proliferated. callus cultures from mutant seeds can be established. The biocompatibility of these cell cultures can be tested with 10 different commercially available hydrogels, including both naturally developed derived polymer gels, such as alginate, and synthetic polymers such as poly(ethylene glycol) diacrylate (PEGDA). Different hydrogel concentrations can be developed in culture media, autoclaved and poured in 6-well plates under sterile conditions. The solid cell cultures can be monitored over a 15 day period for cell viability and proliferation. Findings can indicate that greater hydrogel rigidity can adversely impact biocompatibility, which can prompt to test a range of hydrogel concentrations to determine the ideal stiffness for each type. Agarose, alginate, Gelzan, and Pluronic can emerge as the hydrogels most conducive to plant cell growth. ARA callus cells can be successfully encapsulated in alginate hydrogels, with a demonstrated growth over a 15-day period. In addition, an initial screening/first approximation of the mechanical properties of selected cell-laden hydrogel specimens can be performed via dynamic mechanical analysis (DMA). The storage modulus (G′) can be measured and an increased G′ in cell-laden hydrogels (˜8 kPa) can be observed compared to the controls (˜3 kPa). Hydrogels containing mutant ARA cells of higher lignin content can display even higher values (˜11 kPa).

3D Bioprinting of Developed Cell Cultures & Incubation of Bioprinted Constructs. 3D Bioprinting Provides a Controllable Method to Fabricate Complex Structures that can Enable In Vitro Plant Tissue Generation.

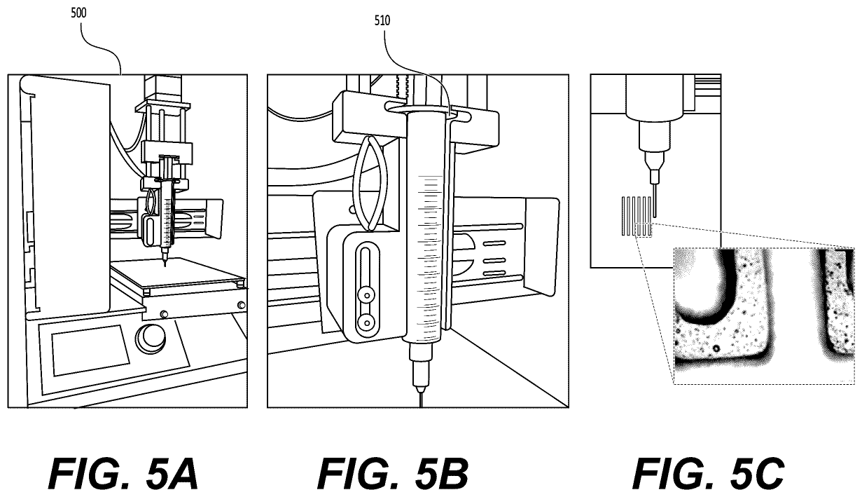

CMCs and ARA cells, developed as described above, can be encapsulated in hydrogels enriched with nutrients, carbohydrates, and plant growth regulators to develop suitable bioinks. FIG. 5A shows an exemplary extrusion-based bioprinting device 500 which can be configured to employ the bioprinting methods described herein. As shown in FIG. 5B, bioinks can be placed in 10 mL sterile luer-lock syringes 510 and printed in 3D scaffolds using an extrusion-based bioprinting method, which allows for the spatial patterning of cell-laden hydrogels using a computer-aided layer by layer deposition technique. FIG. 5C shows the bioprinting device mid-process, such that the bioink is being bioprinted to create a scaffold, or structure. FIG. 5C also illustrates a zoomed-in image of the cells within the bioprinted structure, such that the wood forming plant cells can begin to differentiate after being bioprinted into the structure. Both the bioink and the bioprinting process itself may affect the developed 3D cell microenvironment. To develop suitable 3D microenvironments for cell differentiation and wood formation, bioink formulation and bioprinting process parameters can be optimized.

The desired set of process parameters can be selected from a group consisting of wood forming plant cell type, hydrogel type, incubation time, bioprinting humidity, bioprinting pressure, bioprinting velocity, bioprinting temperature, infill density, and bioprinting needle tip diameter. The desired set of process parameters, as will be appreciated herein, can include the bioprinting process parameters. Values for the selected parameters, as will be described in greater detail herein, can be determined via a surrogate MLM. The desired set of process parameters, in some embodiments, can be selected based at least in part on desired physical properties. In an example embodiment, the desired set of process parameters can be selected based on the desired shape of the structure. In another example embodiment, the desired set of process parameters can be selected based at least in part on the intended application of the generated wood, as will be appreciated herein.

Commercial application of some embodiments disclosed herein include exploring hydrogels, including both naturally derived polymer gels, such as alginate, gelatin (GelMA), and low melting agarose, and synthetic polymers, such as poly(ethylene glycol) (PEG) and its derivatives, which have demonstrated good cell biocompatibility. Hybrid options combining both natural and synthetic polymers can be also considered. The selected hydrogels can be assessed to optimize bioink printability, bioactivity, functionality, and initial mechanical robustness of the bioprinted constructs until cells' differentiation and wood formation occurs. The relevant parameters to be explored and optimized are presented in Table 1.

Once the bioink formula is optimized for a particular application, bioprinting process variables, i.e., extrusion pressure and speed, infill density, needle tip diameter, and printing temperature, can be also explored and optimized for cell viability (max printing temperature can be 37° C. to avoid harming the cells). A high-throughput approach can be adopted to assess cell viability at specific time intervals during two weeks of incubation via bright field microscopy and confocal imaging. For this optimization process, bioink droplets can be printed inside eight-well-chambered coverslips, facilitating cell imaging at multiple time points. Once the process parameters are optimized for cell viability, the bioink can be printed inside petri dishes in mm scale beams of rectangular cross-sections to allow for mechanical testing. More complex 3D structures can be also printed for demonstration purposes.

The CMC and ARA cells may not be immediately available, as they can be developed at a later stage. The bioink formulation and 3D bioprinting process optimization, however, can start at the project's outset, using commercially available ARA cells to ensure high cell viability rates. The optimized bioink formulation and process parameters can be further verified and adjusted in the second year of the study, once the developed CMC and ARA cells are ready. Experiments can be carried out in a laminar flow hood under sterile conditions. The hydrogels can be prepared and autoclaved according to manufacturers' instructions and stored at suitable temperatures to prevent solidification before use. Concentrated liquid cell cultures—including culture media and growth regulators (e.g., auxin and cytokinin)—can be gently mixed with the prepared liquid hydrogels to prepare the bioink. The cell-laden bioinks can be used directly after preparation, and printed inside petri dishes, while executing a G-code file with the geometry of the printed object and the associated printing parameters. Curing/crosslinking strategies can follow based on their impact on cells' viability and structure's integrity. After printing, the 3D constructs can be sealed with parafilm and covered with the same culture medium solution used for the bioink preparation. This solution can be replaced substantially every week together with the parfilm coverage allowing for gas exchange. The bioprinted constructs can be incubated in the dark at 22° C. inside temperature and humidity-controlled chambers for 1-3 months. During the incubation period cell growth and differentiation inside the bioprinted constructs can be examined via imaging (e.g., confocal, TEM) and X-ray computed tomography (CT) (e.g., contrast-enhanced 3D micro-CT scanning).

TABLE 1
Hydrogel selection parameters to optimize the developed bioink.

| Hydrogel | Parameters to be explored for hydrogel selection & bioink |
|---|---|
| features | development |
| Printability | Viscosity: too viscous hydrogels include high pressures which may |
| | damage cells. In contrast, low viscosities may hamper structural |
| | integrity & resolution of printed structures. |
| | Cell density: starting with relatively low densities ~10⁶ cells/ml |
| | and adjust accordingly. |
| | Crosslinking/curing mechanisms: short exposure to crosslinking |
| | agents, UV light and or reversible thermal gelation options can be |
| | Print supports: printing of support materials, e.g., Pluronic F127, to |
| | provide temporary stability before bioink curing can be considered. |
| | Shrinkage, shear-thinning behavior, and sol-gel. |
| Bioactivity | Gel chemistry: impacts of gel's chemistry (e.g., pH) on cells can be |
| (biocompatibility, | examined to avoid cytotoxicity (target: >85% cell viability). |
| proliferation & | Porosity/mass transport: continuous exchange of O2 and CO2, |
| viability) | nutrients, proteins, growth regulators & waste products could be |
| | Gelation temperature: printing/gelation temperatures below 37° C. |
| | can be only considered to avoid harming the cells. |
| Functionality | Insolubility in the cell culture medium can be avoided. |
| | Compatibility with culture medium & growth regulators is needed. |
| Mechanical | Stiffness & strength: mechanical properties influence cell |
| robustness | adhesion, proliferation, and differentiation; printed constructs |
| | could remain structurally intact after printing. |

### Advanced Characterization of the 3D Bioprinted Constructs.

To measure and optimize the mechanical properties of the 3D bioprinted constructs, high-throughput mechanical characterization and microstructural investigations can be performed.

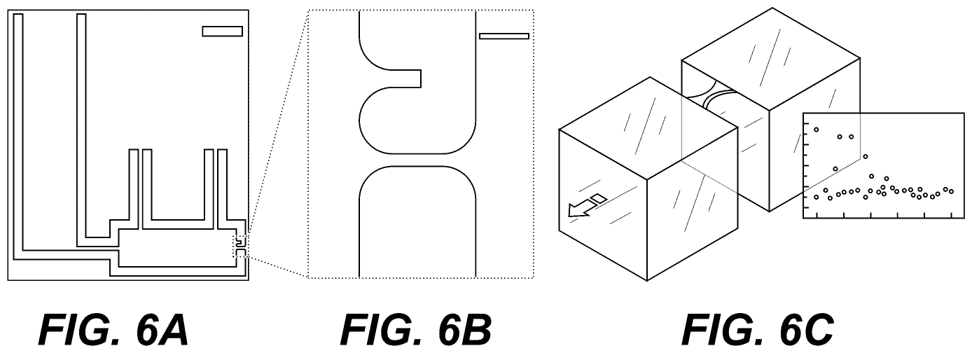

High-throughput mechanical testing: A huge parameter space of growing factors and incubation conditions together with bioprinting parameters can be explored in order to obtain the material with the optimal mechanical properties (i.e., elastic modulus, yield strength, fracture strength and hardness). The produced wood (derived from bioprinting cells) and plant-based test specimens (derived from bioprinting cells) can be in the mm range to allow for high-throughput investigation of growing and printing conditions. However, ASTM Standards and traditional methods for characterizing elastic modulus, yield and fracture strengths do not exist at this length scale. To effectively investigate process/structure/property relationship at the length scale, the PI has developed a high-throughput small scale mechanical testing methodology. Employing such methodology allows for testing tenths of mm scale 3D bioprinted specimens in parallel. The mechanical tests involve applying controlled displacements and identifying the material response of each specimen until failure using a wide-field camera. The controlled displacements are introduced in a contactless manner, by a femtosecond laser as shown in the experimental setup of FIGS. 6A-C. Reflection photoelasticity can be used for measuring the strains after the application of a thin photoelastic coating (PS-1 from Micromeasurement) on the test specimens. The photoelastic recordings offer precise full-field strain information of multiple test specimens in parallel. Hardness measurements can be also performed following the ASTM D143-22 method.

The resultant generated wood can have a plurality of physical properties. In some embodiments, the values of the plurality of physical properties can vary. For example, the high throughput mechanical testing processes, as described herein, can be used to determine the values of the physical properties of the generated wood. The one or more physical properties can include, but are not limited to, tensile strength, flexural strength, yield strength, density, hardness, compressive strength, or any combination thereof. According to an example embodiment, the generated wood can have a density greater than 500 kg/m³, greater than 600 kg/m³, greater than 700 kg/m³, greater than 800 kg/m³, greater than 900 kg/m³, or greater than 100 kg/m³. In some embodiments, the generated wood can have a density of no more than 2,500 kg/m³, no more than 2,400 kg/m³, no more than 2,300 kg/m³, no more than 2,200 kg/m³, no more than 2,100 kg/m³, no more than 2,000 kg/m³, no more than 1,900 kg/m³, no more than 1,800 kg/m³, no more than 1,700 kg/m³, no more than 1,600 kg/m³, no more than 1,500 kg/m³, no more than 1,400 kg/m³, no more than 1,300 kg/m³, no more than 1,200 kg/m³, no more than 1,100 kg/m³, or no more than 1,000 kg/m³. In some embodiments, the generated wood can have a range of densities including any of the above values for upper and lower limits, e.g., 500-2500 kg/m³, 900-1300 kg/m³, or 1,000-2,000 kg/m³. According to an example embodiment, the generated wood can have a tensile strength greater than 80 MPa, greater than 90 MPa, greater than 100 MPa, greater than 110 MPa, greater than 120 MPa, greater than 130 MPa, greater than 140 MPa, or greater than 150 MPa. In some embodiments, the generated wood can have a tensile strength of no more than 300 MPa, no more than 290 MPa, no more than 280 MPa, no more than 270 MPa, no more than 260 MPa, no more than 250 MPa, no more than 240 MPa, no more than 230 MPa, no more than 220 MPa, no more than 210 MPa, no more than 200 MPa, no more than 180 MPa, or no more than 160 MPa. In some embodiments, the generated wood can have a range of tensile strengths including any of the above values for upper and lower limits, e.g., 80-300 MPa, 100-200 MPa, or 90-250 MPa.

Microstructural imaging & material characterization: Microstructural characterization of the specimens exhibiting best the mechanical properties can be conducted. Confocal laser scanning and electron microscopy methods can be employed to image the microstructure, measure cell dimensions, determine the cellulose microfibril angle, and identify potential residual hydrogel. The test specimens can be embedded in paraffin wax, while sectioning of the sample can be done by hand-held microtome. For the confocal microscopy the sample can be stained with fluorescent cell wall markers. Finally, the bioprinted construct density (ratio of dry mass divided by its volume) can be evaluated.

Risks and Mitigation: The orientation of the TEs can cause the bioprinted construct to exhibit different properties in different directions, while specimens can behave radically differently with minor changes in loading conditions. To mitigate these risks testing can be carried out at multiple axes, and dynamic nanoindentation can be employed to extract time-dependent mechanical properties.

### Process Scale-Up Via Machine Learning (ML)-Based Modeling & Experiment Automation.

In order to further understand and optimize the process/structure/property relationship between CMCs and 3D bioprinted structures, and to enable manufacturing scalability and future predictions and translation of the developed in vitro wood forming method in other tree species, the present disclosure develops relevant process diagnostics and automation. Data collection from, and automation of various parts of the disclosed wood manufacturing process (from culture media preparation to bioprinting to incubation to mechanical characterization), can enable the development of a multiscale biomechanical model, where the complex microscale features and interactions can be linked to the bioprinted constructs' macroscopic behavior in a future FMRG. In this present disclosure the present disclosure can build the foundations for this model, through the two tasks described below, which can run in parallel with.

ML-based surrogate model for predicting biomechanical behavior: The microscale cell interaction and the forces generated inside the cell-laden hydrogel scaffolds during wood formation significantly affect the macroscale evolution and performance of the 3D bioprinted structures. However, these microscale interactions are complex, changing over time, and cannot be directly linked to the structures' macroscopic behavior in an analytical way. Therefore, understanding these interactions at both the microscopic and macroscopic levels is crucial for scaling-up the disclosed wood manufacturing process and gaining a comprehensive understanding of it. Factors such as cell stiffness, size, and shape, and bioprinting process parameters, like pressure, speed, and needle tip diameter, can significantly alter the mechanical properties of the bioprinted constructs. To explore this complex parameter space, the present disclosure can adopt an ML surrogate approach. Our surrogate involves training supervised ML algorithms with input data from images obtained from confocal microscopy at various steps in the incubation process, selected incubation protocols, and output data representing the final mechanical properties of the produced 3D bioprinted constructs (stress vs. strain data and hardness data). Some embodiments can use convolutional neural networks (CNNs) building upon a ML framework. The ML surrogate can serve a simple multiscale model to predict structural performance of the developed in vitro wood.

Experiment automation: The replacement of the syringe system of the 3D bioprinter with an automated syringe loading mechanism for faster and reliable bioprinting can be explored. A modular bioink refill system, already developed at co-PI Aidun's lab, consisting of a level sensor, a peristaltic pump, and a temperature sensor can be incorporated and adjusted to the optimized bioprinting process variables. This modular system can also monitor and regulate the temperature of cell culture media to ensure consistent and optimal conditions. During the incubation period of the 3D bioprinted constructs, the climatic conditions (i.e., temperature, humidity, CO2 levels, air pressure) inside the incubator chambers can be continuously monitored and regulated, by employing an automated climate control system developed at Aidun's lab. Future scale-up of the disclosed manufacturing process could include automated cell culture development in bioreactors combined with robotic arms for 3D bioprinting and testing but is out of the scope of this present disclosure.

Metrics: Accuracy of the surrogate model via testing with experimental data; Monitoring and regulating temperature of cell culture media; Tuning bioink loading; Monitoring/regulating climate conditions during incubation of 3D bioprinted constructs. Risks and Mitigation: Insufficient amount of experimental data (relative to the datasets for CNN training) may restrict the predictive performance of the surrogate model. To avoid this risk, a multiscale biomechanical model can be coupled with the CNN model to reduce the training data.

### Enabling Future Manufacturing

A thriving future US manufacturing demands feedstocks and products that offer improved quality, lower production costs, and sustainable supply chains. These can create new opportunities in emerging markets, such as construction, energy storage, and space exploration, and can reduce environmental impacts, while supporting local and circular economic models. In this context, this present disclosure aims to 1) gain insights into the systematic and de novo growth of a first-of-its-kind in vitro wood material of high-performance, and 2) disseminate findings to promote the development of a technically agile manufacturing workforce.

The adoption of this in vitro wood approach can disrupt timber and wood manufacturing sectors, allowing for local, on demand wood production of desired shapes while unburdening forests. This project can enable a new class of lab-grown, renewable materials with improved mechanical properties compared to their natural counterparts, coupling synthetic biology of plant cells with advanced manufacturing. The disclosed manufacturing capability stands in contrast with current, polluting, and wasteful wood production pathways, or state-of-the-art 3D printing techniques which formulate synthetic wood-polymer composite parts through a multistep process. Rather, this approach advances knowledge in the current plant culture, gene editing and 3D bioprinting research space, and ushers in a new manufacturing field that could be implemented for on-demand wood production, independent of climatic conditions and land availability.

The major challenges that can be overcome to enable in vitro wood manufacturing concern establishing suitable conditions to mimic the natural plant cell microenvironment to induce wood formation. Wood formation is still an intricate and enigmatic process comprising numerous parameters which can be orchestrated in a specific manner. a transdisciplinary team can leverage latest advancements in plant biology, additive manufacturing, and advanced characterization to capture and coordinate at least a portion of these parameters to produce in vitro wood, and systematically address wood process-structure-property relationships. Although high risks are entailed, if successful this project can have revolutionary implications in timber and wood products manufacturing. Furthermore, the gained insights can be expanded to transform other manufacturing domains, including the development of high-performance lattice structures, green microelectronics, and in-space manufacturing.

Manufacturing of renewable, plant-based materials is an emerging field attracting attention globally, with several leading countries making strides in this area. Canada (e.g., British Columbia BioProducts Institute) is investing in research and development of new bioproducts from wood, including cellulose-based materials that could be used in textiles and composites. China is exploring the use of plant biotechnology to produce wood-like materials using genetically modified microorganisms (e.g., The Center for Synthetic and Systems Biology at Tsinghua University). China is also investigating the use of lignin, a waste product of the paper industry, to create new materials that could replace plastic in various applications (Beijing Key Laboratory of Lignocellulosic Chemistry). In Europe, researchers are developing new techniques to produce high-performance wood materials, including using nanocellulose to enhance the strength and durability of wood products (e.g., The Synthetic Biology Group at ETH Zurich). Investing in in vitro wood production can be a strategic move for the US to maintain its leadership in the global timber manufacturing industry, and to advance in value-added forest products and advanced biomanufacturing.

In this description, numerous specific details have been set forth. It is to be understood, however, that implementations of the disclosed technology may be practiced without these specific details. In other instances, well-known methods, structures, and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form. By “comprising” or “containing” or “including” is meant that at least the named element, or method step is present in article or method, but does not exclude the presence of other elements or method steps, even if the other such elements or method steps have the same function as what is named.

It is to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Although embodiments are described herein with respect to systems or methods, it is contemplated that embodiments with identical or substantially similar features may alternatively be implemented as systems, methods and/or non-transitory computer-readable media.

As used herein, unless otherwise specified, the use of the ordinal adjectives “first,” “second,” “third,” etc., to describe a common object, merely indicates that different instances of like objects are being referred to and is not intended to imply that the objects so described must be in a given sequence, either temporally, spatially, in ranking, or in any other manner.

While certain embodiments of this disclosure have been described in connection with what is presently considered to be the most practical and various embodiments, it is to be understood that this disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the technology and also to enable any person skilled in the art to practice certain embodiments of this technology, including making and using any apparatuses or systems and performing any incorporated methods. The patentable scope of certain embodiments of the technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A bioink for bioprinting comprising:
a plurality of wood forming plant cells;
one or more hydrogels encapsulating the plurality of wood forming plant cells; and
plant cell culture medium components.

2. The bioink of claim 1, wherein the plurality of wood forming plant cells are cambial meristematic cells (CMCs).

3. The bioink of claim 2, wherein the CMCs are derived from Populus sp. trees, Pinus taeda trees, Paulownia sp. trees, or any combination thereof.

4. The bioink of claim 1, wherein the plurality of wood forming plant cells comprises genetically modified plant cells.

5. The bioink of claim 4, wherein the genetically modified plant cells comprise one or more of lignin mutant plant cells, hemicellulose mutant plant cells, and cellulose mutant plant cells.

6. The bioink of claim 1, wherein the one or more hydrogels comprise alginate, gelatin (GelMA), agarose, Gelzan, poly(ethylene glycol) (PEG), pluronic, Carboxymethyl Cellulose, or any combination thereof.

7. The bioink of claim 1, wherein the plant cell culture medium components comprise nutrients, carbohydrates, growth regulators, or any combination thereof.

8. The bioink of claim 1, wherein the bioink is configured to be loaded into a bioprinting system.

9. The bioink of claim 1, wherein the bioink comprises a bioink formula, wherein the bioink formula is optimized using a surrogate machine learning model (MLM).

10. A method of generating wood in vitro comprising:
culturing a plurality of wood forming plant cells;
encapsulating the plurality of wood forming plant cells in one or more hydrogels to form a plurality of encapsulated wood forming plant cells;
developing one or more bioinks from the plurality of encapsulated wood forming plant cells, the one or more bioinks further comprising one or more plant cell culture medium components; and
generating structures comprising the one or more bioinks.

11. The method of claim 10, further comprising:
incubating the structures to induce wood formation.

12. The method of claim 11, further comprising monitoring the incubated structures via imaging, confocal imaging, X-ray computed tomography, contrast-enhanced 3D micro-CT scanning, or any combination thereof.

13. The method of claim 11, further comprising determining one or more physical properties of the incubated structures, wherein the one or more physical properties are selected from the group consisting of tensile strength, flexural strength, yield strength, density, hardness, compressive strength, and impact strength.

14. The method of claim 13, further comprising determining, based at least in part on the one or more physical properties, a desired set of process parameters for generating wood.

15. The method of claim 14, wherein the desired set of process parameters comprises one or more selected from the group consisting of wood forming plant cell type, hydrogel type, incubation time, bioprinting humidity, bioprinting pressure, bioprinting velocity, bioprinting temperature, infill density, and bioprinting needle tip diameter.

16. The method of claim 14, wherein values of the desired set of process parameters are determined via a surrogate machine learning model (MLM), wherein the surrogate MLM is trained based at least in part on one or more of images obtained during an incubation process and one or more physical properties of the generated wood.

17. The method of claim 10, wherein the generated wood has a tensile strength of between 80 and 300 MPa.

18. The method of claim 10, wherein generating the structures comprises bioprinting the one or more bioinks via an extrusion-based method.

19. The method of claim 10, wherein the structures are generated with a predetermined shape based on an intended application of the generated wood.

20. The method of claim 10, wherein the one or more hydrogels have a storage modulus of between 3 and 5 kPa.
